(19) United States
(12) Patent Application Publication
(10) Pub. No.: US 2007/0197878 A1
(43) Pub. Date: Aug. 23, 2007

(54) Wearable device, system and method for monitoring physiological and/or environmental parameters

(76) Inventor: Dror Shklarski, YAVNE (IL)

Correspondence Address: EMPK & SHILOH, LLP, 116 JOHN ST,, SUITE 1201, NEW YORK, NY, 10038, US

(21) Appl. No.: 11/631,948

(22) PCT Filed: Jul. 10, 2005
(86) PCT No.: PCT/IL05/00733

§ 371 Date: Jan. 9, 2007

Related U.S. Application Data
(60) Provisional application No. 60/586,243, filed on Jul. 9, 2004.
(60) Provisional application No. 60/586,242, filed on Jul. 9, 2004.
(60) Provisional application No. 60/589,599, filed on Jul. 21, 2004.

(51) Int. Cl. A61B 5/00; G06Q 10/00; G06Q 50/00

(52) U.S. Cl. 600/300; 128/903; 705/2

(57) ABSTRACT

A system, device and method are provided for monitoring parameters, comprising a wireless mobile monitoring device including an array of sensors; and a medical center server enabled to remotely reconfigure the functioning of the monitoring device. In some embodiments the system may be remotely customized by the server. The array of sensors may include one or more physiological sensors and/or one or more environmental sensors. The monitoring device may function in keeper mode, extended mode, and/or emergency mode, and may enable measurement of one or more selected parameters continuously and/or intermittently. In some embodiments the communications between the monitoring device and the medical center are encrypted and/or authenticated.

### FIELD OF THE INVENTION

The present invention relates to wearable devices, systems and methods for monitoring physiological and/or environmental parameters, and to communication devices, systems and methods for monitoring selected physiological and/or environmental parameters by the wearable devices, remotely responding to monitored parameters, and remotely updating the wearable devices.

### BACKGROUND OF THE INVENTION

Continuously monitoring a patient's physiological condition generally requires the patient's hospitalization, usually at great cost, especially where long term monitoring is required. In certain situations it is possible to monitor the physiology of patients who are physically outside of the hospital using wearable monitoring devices.

There are, for example, wrist-worn devices that typically record a patient's physiological data, such as the patient's ECG, during a predetermined recording time. These devices may include event recorders that may capture a patient's physiological data during a physiological “event”, such as a cardiac arrhythmia or an episode of patient discomfort. The event recording may be activated manually by the patient or automatically by when predefined event criteria are met.

Wrist-worn devices typically require that a patient return to a medical center periodically or remotely communicate with a medical center in order to transfer the recorded data for analysis, interpretation and/or treatment by medical staff.

### SUMMARY OF THE INVENTION

A system, device and method are provided for monitoring parameters. According to some embodiments of the present invention, the monitoring system may include a wireless mobile monitoring device including an array of sensors; and a medical center server enabled to remotely reconfigure the functioning of the monitoring device. In some embodiments the system may be remotely customized by said server. The array of sensors may include one or more physiological sensors and/or one or more environmental sensors. The monitoring device may perform one or more functions, including selected from the group consisting of measuring parameters, transmitting parameter data, processing parameter data, analyzing parameter data, initiating device actions, updating parameter settings, providing warnings, and providing instructions. The monitoring device may function in keeper mode, extended mode, and/or emergency mode, and may enable measurement of one or more selected parameters continuously and/or intermittently. In some embodiments the communications between the monitoring device and the medical center are encrypted and/or authenticated.

According to some embodiments of the present invention, a device for monitoring of parameters is provided, the device including an array of sensors, each sensor having a sensor controller; and a main controller to enable reconfiguration of the sensor controllers by commands received from a remote server.

According to some embodiments of the present invention, a method is provided for remotely reconfiguring a monitoring device, the method comprising transmitting commands to a wireless monitoring device, from a medical center server, to remotely reconfigure settings of the device; and reconfiguring settings of the device, by a main controller in the wireless monitoring device.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of a medical monitoring system according to some exemplary embodiments of the present invention;

FIGS. 2A, 2B, and 2C are schematic illustrations of external top, bottom, and side view layouts, respectively, of a wearable device according to some exemplary embodiments of the present invention;

FIG. 3 is a schematic illustration depicting an internal layout of a wearable device according to some embodiments of the present invention;

FIG. 4 is a schematic flow-chart illustrating a method of wirelessly updating diagnostic ranges of a wearable device according to some exemplary embodiments of the present invention; and

FIG. 5 is a schematic flow-chart illustrating a method of wirelessly requesting data for selected physiological and/or environmental parameters from a wearable device, according to some exemplary embodiments of the present invention.

### DETAILED DESCRIPTION OF THE INVENTION

The subject matter regarded as the invention is particularly pointed out and distinctly claimed in the concluding portion of the specification. Embodiments of the invention, however, both as to organization and method of operation, together with objects, features and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or to a similar electronic computing device, that manipulates and/or transforms data represented as physical, such as electronic quantities within the computing system's registers and/or memories into other data similarly represented as physical quantities within the computing system's memories, registers or other such information storage, transmission or display devices.

The processes and displays presented herein are not inherently related to any particular apparatus. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of embodiments of the invention as described herein.

It should be appreciated that according to some embodiments of the present invention, the method described below, may be implemented in machine-executable instructions. These instructions may be used to cause a general-purpose or special-purpose processor that is programmed with the instructions to perform the operations described. Alternatively, the operations may be performed by specific hardware that may contain hardwired logic for performing the operations, or by any combination of programmed computer components and custom hardware components.

Although the scope of the present invention is not limited in this respect, the wearable device disclosed herein may be implemented in any suitable wired or wireless device that may be a handheld, worn, or other suitable portable communications device. By way of example, the wearable devices may include wireless and cellular telephones, smart telephones, personal digital assistants (PDAs), wrist-worn devices, and other suitable wearable devices or any parts of them. Alternatively, according to other embodiments of the present invention, the system and method disclosed herein may be implemented in computers.

Embodiments of the present invention are directed to improved wearable devices, systems, and methods for monitoring vital parameters. Monitoring, as used hereinafter, may refer to various functions associated with intelligent or smart monitoring of one or more parameters associated with a patient, for example, measuring physiological and/or environmental parameters, transmitting data, receiving data, processing, analyzing and/or evaluating data, providing warnings, advice and/or treatment instructions, updating monitoring thresholds and/or device functions etc. For example, some embodiments may enable monitoring of physiological and/or environmental parameters, sending alerts to a Medical Center (MC) or to a patient, data processing of sensed data, and updating device parameters and/or functions.

In other embodiments the devices, systems and methods described below may enable remotely initiating measurements, for example, by providing instructions from a MC to initiate a selected operations or actions by the wearable device, as is described in detail below. The wearable device, according to some embodiments of the present invention, may be operated in accordance with different modes of operation, wherein the different modes may be configured based, for example, on the health conditions of individual patients. The wearable device, according to other embodiments of the present invention, may be remotely requested to take measurements of physiological and/or environmental parameters, or to initiate other suitable functions. For example, a server associated with a medical center (MC) may initiate one-time operations, operations that occur multiple times, permanent operations, temporary operations etc. Such requests, which may include providing software updates, may be initiated by the MC in accordance with previous measurements received from the wearable device. The request may be done by the MC staff, for example, medical staff, information technology staff and/or technical/engineering staff. In other embodiments a device at the MC may automatically update various parameters in accordance with pre-configured criteria.

The wearable device according to some embodiments of the present invention, may independently transfer a patient's physiological and/or environmental data to, for example, a MC, when certain parameters are above or below predetermined ranges or thresholds that may be defined according to the particular needs of the patient. In some embodiments, the wearable device may also transfer the data to the MC if the parameters are within predetermined thresholds or ranges. In some embodiments, the MC may receive, via a communications channel, a patient's physiological and/or environmental data and additional information, such as, for example, the location of the patient, directly from the wearable device. The MC may remotely update, for example, the ranges and/or thresholds for determining the status of vital parameters of an individual patient. For example, in one embodiment an update may be initiated at the discretion of staff in the MC, for example, medical, information technology, engineering or technical staff, etc., or automatically by a suitable device and/or programmed computer, which may include hardware and/or software, at the MC itself or remotely connected to the MC. The MC may remotely update operational features, for example, changing modes of operation, adding new features, updating the device's software and/or part thereof, for a wireless device of an individual patient or for a group of patients.

In another embodiment a person or persons at the MC may remotely determine, for example, the need for measurements and/or updated measurements of physiological and/or environmental parameters of an individual user, or a group of users, at the discretion of the staff at the MC, or the MC system automatically. In some embodiments the MC may remotely send messages to the user. In some embodiments the wearable device may perform the requested measurement automatically without the user's intervention. In some other embodiments, the wearable device may request the intervention and/or confirmation of the user before taking measurement(s).

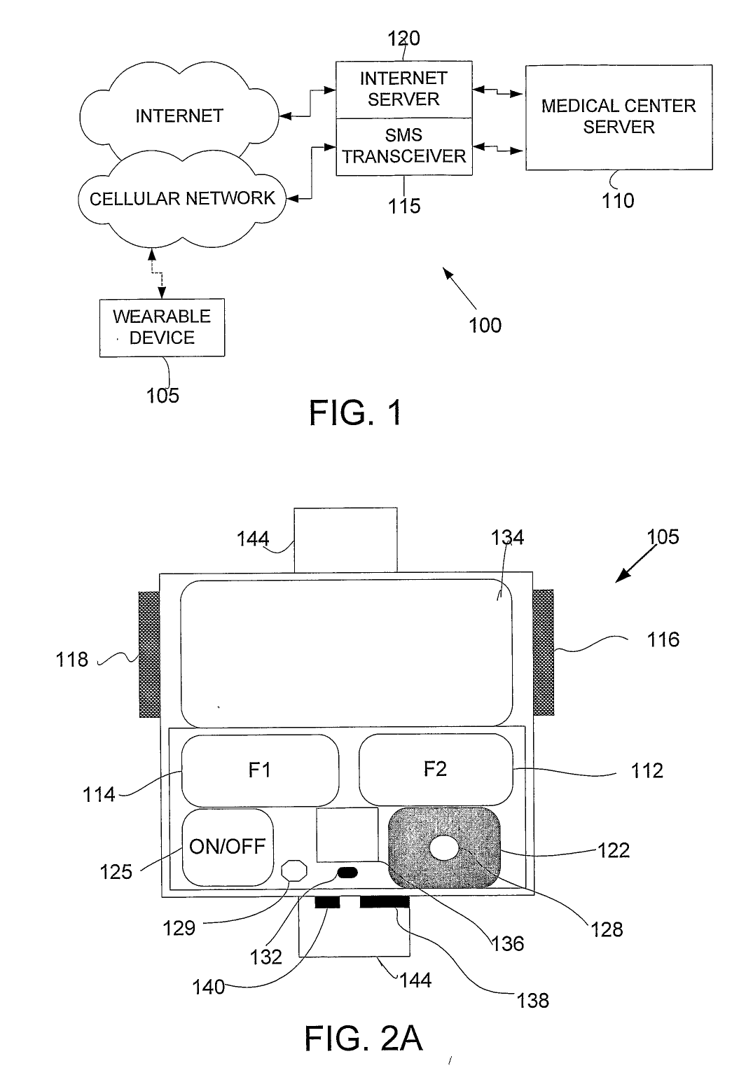

Reference is now made to FIG. 1, which schematically illustrates a medical monitoring system 100 in accordance with some exemplary embodiments of the present invention. Medical monitoring system 100 may include, for example, at least one wearable device 105 that may communicate with a medical center (MC) server 110. Wearable device 105 may have a bidirectional communication link with MC server 110, which may be associated with, for example, a clinic, hospital, remote center, medical professional, or any other suitable provider of suitable medical services. For example, wearable device 105 may communicate with MC server 110 using a serial communication port, a parallel connection, USB, a modem, network card (e.g., ADSL, Cable, satellite) or other data communications technologies. For example, wearable device 105 may communicate with MC server 110 using wireless data communication, for example, using cellular communication (e.g., General Packet Radio Service (GPRS)), satellite communications technology, wireless LAN technology, infrared technology, Wireless Fidelity (WiFi), Bluetooth, or other suitable wireless communications technologies. Data may be transferred between wearable device 105 and MC server 110 using the above or other suitable means.

The communication may be performed over a computer network, for example, the Internet or a local area network (LAN), etc. There may be a plurality of bidirectional and/or uni-directional communication channels between MC server 110 and wearable device 105, and there may be a plurality of medical centers (MC), MC servers 110 and/or wearable devices 105.

In one embodiment the bi-directional communication channel between the MC server 110 and wearable device 105 is a Short Message Service (SMS) channel that may enable communication of data via SMS transceiver 115 to and/or from the wearable device 105, via a cellular communications network. The SMS channel may enable transmission of messages from wearable device 105 to MC server 110, via SMS transceiver 115. In one embodiment the bi-directional communication channel between the MC server 110 and wearable device 105 is an Internet Protocol (IP) based channel, that may enable communication of data via Internet server 120, for example, using File Transfer Protocol (FTP) or other suitable data transfer protocols. In some embodiments a combination of communication networks may be used. For example, if the SMS channel is not available and/or not chosen by the wearable device 105, wearable device 105 may communicate with MC server 110 using FTP. In other embodiments wearable device 105 may communicate with MC server 110 using, for example, SMS and Internet communications. In some embodiments wearable device 105 may communicate with MC server 110, via a Web interface, for example, a Website, where data, commands, and/or requests etc. may be entered and/or received by wearable device 105 and/or MC server 110.

In one embodiment the bidirectional communication channel between the MC server 110 and wearable device 105 may utilize TCP/IP protocol. In one embodiment a File Transfer Protocol (FTP) may be used to upload physiological data of the patient, e.g., sensed measurement data, from wearable device 105 to MC server 110, and to download data such as updates to software modules from MC server 110 to wearable device 105. Usage of FTP or any other suitable protocol may require the wearable device 105 to logon as an FTP client to the Internet server 120.

In some embodiments a voice channel, as described below, may be used to enable the staff at MC server 110, or a device or suitable software and/or hardware associated with the MC server 100, to communicate with the patient who is using wearable device 105 and/or to enable the patient using wearable device 105 to communicate with the staff of MC server 110 or the MC server 110 itself.

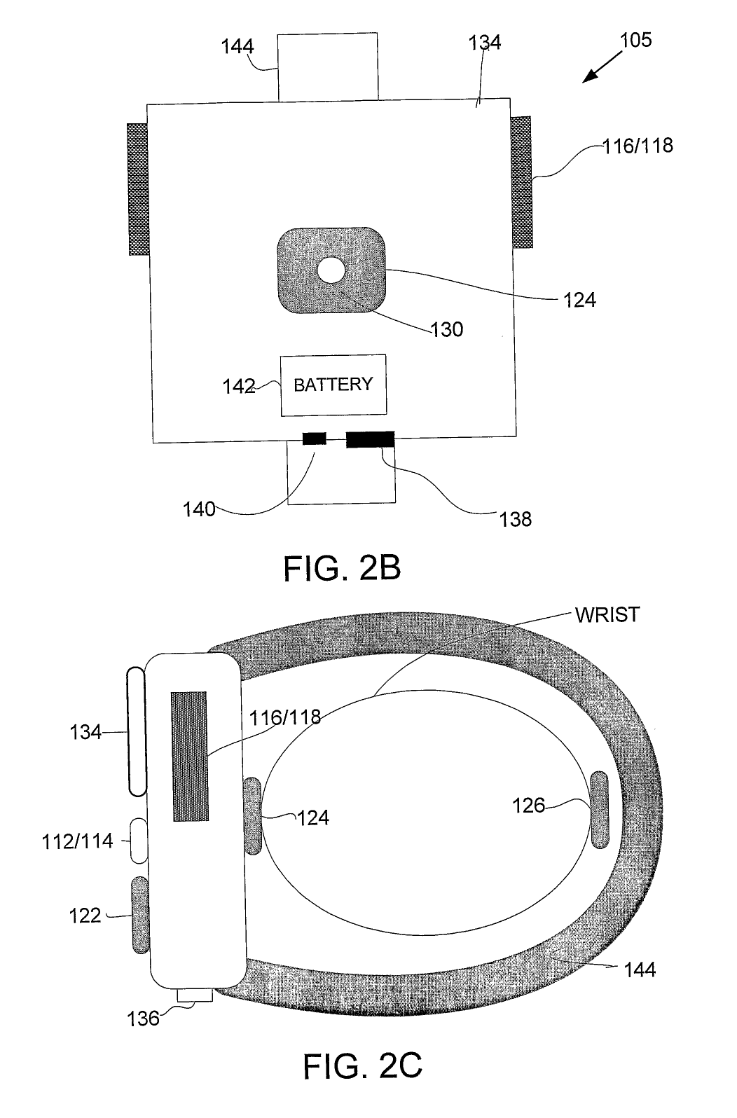

Reference is now made to FIGS. 2A, 2B, and 2C which schematically illustrate external top, bottom, and side view layouts, respectively, of a wearable device 105 in accordance with some exemplary embodiments of the present invention. Wearable device 105 may include, for example, input components such as functional buttons 112 and 114 for inputting data or commands to operate wearable device 105. Wearable device 105 may include, for example, emergency buttons 116 and 118 that may be used to manually initiate an emergency mode (e.g., by pressing them together or pressing one of them), and an On/Off button 125 to switch wearable device 105 on or off. The On/Off button 125 may be unified with any of the other buttons, for example functional buttons 112 and 114. Wearable device 105 may include one or more electrodes, for example, an ECG RA (Right Arm) finger electrode 122, an ECG LA (Left Arm) wrist electrode 124 (FIG. 2B), and an ECG REF (Reference) wrist electrode 126 (shown in FIG. 2C). Electrodes 122 and 124 may be located in any suitable location or locations on wearable device 105. For example, electrode 124 may be located on the top side of wearable device 105. In some embodiments the ECG REF. Wrist electrode 126 may be located at any suitable location in the inner side of wearable device 105 or on the inner side of strap 144. Wearable device 105 may be worn on a patient's left or right hand or arm, e.g., on the wrist, or on the left or right foot or leg, e.g., on the ankle, and the various components may be appropriately located to enable measuring of parameters whether on the left and/or right hand and/or arm and/or foot and/or leg.

In some embodiments ECG electrodes 124 and/or 126 may be used to sense the ECG of the patient, by, for example, performing ECG measurements when the patient touches finger electrode 122 with his/her finger. In addition, wearable device 105 may include a blood oxygen saturation level (SpO₂) transceiver 128 to measure the level of the oxygen in the patient's blood, a pulse transceiver 130 (shown in FIG. 2B) to measure the patient's pulse, and/or a microphone 132 that may be used to enable the patient's voice to be input, and optionally converted to electronic impulses for electronic communication. Blood oxygen level (SpO₂) transceiver 128 may be incorporated, for example, in the ECG RA finger electrode 122 and/or may be a separate sensor. Blood oxygen level (SpO₂) transceiver 128 may be located in a suitable location, for example, in the inner side of the strap 144. In some embodiments wearable device 105 may include a pulse transceiver or sensor 129 located in a suitable position in wearable device 105. In some embodiments, wearable device 105 may include one or more transceivers, electrodes, or sensors to enable measurement of physiological data including, for example, blood pressure data, skin temperature data, respiration data, cardio impedance data, blood sugar or glucose level, and/or other suitable data. In some embodiments, wearable device 105 may include one or more transceivers, electrodes, or sensors to enable measurement of environmental data including, for example, external temperature data, air humidity data, air pollution data, and/or other suitable data. Other suitable sensors, detectors, devices etc. may be used.

Wearable device 105 may include a speaker 136 to enable a patient to receive audio signals, for example voice communication, from MC server 110. When wearable device 105 is operated in a continuous mode of operation, wearable device 105 may, for example, continuously or according to a pre-defined schedule, read the pulse of the patient, using pulse transceiver 130. The location of pulse transceiver 130 within wearable device 105 may be appropriately positioned to enable sensing of the pulse of the patient. Pulse transceiver 130 may be incorporated within electrode 124 or may be separate from electrode 124. An indication of the pulse of the patient and/or other parameters may be presented on the display area 134 of wearable device 105. The pulse and/or other parameters may also be transferred to the MC server 110. Other sensor mechanisms may be used.

Display area 134 may display additional information such as, for example, medical parameters of the patient, messages received from a MC, operational instructions, date and time, parameters that are related to functional elements of wearable device 105 etc. Display area 134 may be, for example, a color display and/or a monochromatic display and may have any desired resolution, depending on the type of data to be displayed. In some embodiments, display area 134 may include an interactive display, for example, a touch sensitive display. Display area 134 may display any combination of alphanumeric characters, and/or text and/or two-dimensional and/or three-dimensional graphics and/or icons.

Additional elements in wearable device 105 may include one or more service connectors, for example, a service connector 138 that may connect the wearable device 105 to external units such as, for example, a computer that may help provide software updates, testing, technical diagnostics etc., a testing unit that may enable testing the usability of device 105, an external medical device, for example, to measure blood pressure, ECG etc., an external display unit, communication unit, for example, a Blue-tooth chip and circuitry, and/or other suitable external units. Wearable device 105 may include a charge connector 140 that may be used to connect wearable device 105 to a power source to enable charging of a battery 142 (FIG. 2B). A charger connector 140 may be included in service connector 138. Wearable device 105 may include optional strap 144 that may be used to attach wearable device 105 to the wrist or other location of the patient. Wearable device 105 may include various other suitable components and/or devices, which may be implemented using any suitable combination of elements and components and may incorporate hardware and/or software.

In accordance with some embodiments of the present invention, medical monitoring system 100 may operate in at least one of keeper mode, extended mode, and emergency mode, or any other appropriate mode, as described below.

The keeper mode may be used as the default mode of wearable device 105, such that wearable device 105 may enter this mode when the device is switched on. Other modes may alternatively be used as the default mode. In the keeper mode, wearable device 105 may, for example, continuously or intermittently, read the pulse and/or another parameters of a patient. In one example of keeper mode functioning, wearable device 105 may display parameter data on display area 134, may alert the patient with a message on display area 134, and/or may alert the patient using an audible signal via speaker 136, for example, by playing back predefined audio signals. In addition, wearable device 105 may transmit the measured parameters to MC server 110 for analyses or processing of the measured parameters, for example, using a FTP channel and/or a SMS channel. In the event where the staff in MC server 110 determines that the patient's pulse is abnormal, according to predetermined criteria or ranges described in detail below, wearable device 105 may alert the patient.

According to some embodiments of the present invention wearable device 105 itself may determine when one or more parameters are abnormal or, for example, in a danger range, instead of or in addition to the staff in MC server 110. According to some embodiments of the present invention MC server 110 may automatically determine when one or more parameters are abnormal or, for example, in a danger range, instead of or in addition to the staff in MC server 110. Additionally, wearable device 105 may send a warning message to MC server 110, using, for example, the SMS channel, FTP channel etc. When wearable device 105 is operated in keeper mode, parameters such as pulse, SpO₂, and ECG may be monitored continuously and/or at selected intervals, for example, every twelve hours.

In the extended mode, wearable device 105 may be set to perform operations according to a pre-defined schedule, for example, to periodically measure oxygen levels in the patient's blood (SpO2) and/or ECG. In this mode, wearable device 105 may display parameter data on display area 134, may alert the patient with a message on display area 134, and/or may alert the patient using an audible signal via speaker 136, for example, by playing back predefined audio signals. In addition, wearable device 105 may transmit the measured parameters and/or results from analyses or processing of the measured parameters, to MC server 110, for example, using FTP channel and/or SMS channel. When wearable device 105 is operated in extended mode, vital signs such as pulse, SpO2, and ECG, may be monitored, for example, five times a day by default (e.g., the default may be at shorter or longer intervals, as required). If the staff at MC server 110 or the MC server 110 detect, for example, that the heart rate, oxygen level in the blood, and/or ECG records and/or other data are abnormal (e.g., according to pre-defined criteria or ranges as discussed below), wearable device 105 may alert the patient by providing output signals in the display area 134 or via speaker 136. Additionally or alternatively, wearable device 105 may send a message to MC server 110, or to another destination, for example, using the FTP channel.

In emergency mode a patient may initiate operation of the medical monitoring system 100 by pressing, for example, any of the emergency buttons 116 or 118. When operating in emergency mode, wearable device 105 may send emergency messages to MC server 110 or to another destination using, for example, the FTP channel. Emergency messages may additionally or alternatively be sent to MC server 110 or to another destination via the SMS channel, for example, in cases where the FTP channel is not available. In addition, when entering an emergency mode, measurement of SpO2 level, ECG level, and/or additional suitable parameters may be initiated. The staff of MC server 110 or the MC server 110 itself may initiate a call to the patient of wearable device 105, or may send a message etc.

According to some embodiments of the present invention, software or device program updates (referred to herein as “software updates”) may be implemented to enable individualized adaptation of operation parameters of device 105. Customizable software updates may include, for example, customizing one or more modes of operation for each patient, customizing ranges or thresholds for monitoring of an individual patient's parameters, customizing timing of parameter measurements, customizing alert functions, determination of types of measurements to be monitored, customizing diagnostic ranges, adding new features or software improvements, deleting features that are not relevant for a particular patient's condition monitoring, customizing operational modes, correcting software problems, and/or any other suitable modifications. The customized or individualized programs may be programmed directly into wearable device 105 using wire based or wireless data communication, and/or may be remotely transferred to wearable device 105. In this way the timing parameters or other aspects of operation of wearable device 105 may be modified and updated, optionally remotely, by MC server 110, at the discretion of the MC staff or automatically using pre-defined criteria. For example, the MC may remotely initiate a certain mode of operation for wearable device 105, and/or may remotely change vital parameter ranges, etc., optionally for each patient individually. Customized or individualized programs may be programmed directly into a single wearable device 105 or into a group of wearable devices 105.

In accordance with some embodiments of the present invention, wearable device 105 may be able to receive SMS messages, for example, from the MC server 110 via the SMS channel. The SMS messages may be displayed to the patient on display area 134. The SMS messages may be selected from a list of pre-defined messages or written by the staff in MC server 110. SMS messages may include instructions to perform additional tests, embedded or attached software updates, instructions to logon to Internet server 120 for software or device program updates, alerts and/or instructions for the patient to physically visit the MC, updated medical parameters or diagnostic ranges, or any other suitable data.

In some embodiments of the present invention, diagnostic ranges of the wearable device 105 may be defined for an individual patient, to facilitate the monitoring and/or analysis of the sensed vital signs of wearable device 105. For example, the MC staff or an automatic procedure or device connected with the MC server 110 may initiate diagnostic modifications to help determine a patient's status, for example, to enable remote testing of the patient's vital signs, and/or to command the medical monitoring system 100 to operate in desired modes of operation, etc. New ranges, commands, etc., may be determined for each patient by the MC staff or by an automatic procedure executed by a device at the MC, and may be programmed into the wearable device 105 either by wire-based or wireless data communications.

In accordance with some embodiments of the present invention, as discussed in detail below, various types of diagnostic ranges may be defined, for example: a “normal” range; a “deviation” range; and a “risk” range. The normal range may be defined as the range of the medical parameters of the patient when the patient is in his/her normal medical condition, for example, as determined by an initial medical examination, or as according to a later updated. When measurements are within the normal range, wearable device 105 may record and store the results, and may transmit the data to MC server 110, for example, once a day or in accordance with any suitable preset schedule. The timing and/or frequency of the communications between MC server 110 and device 105 may be modified remotely by MC server 110, or locally by the patient, or by any other suitable means.

A deviation range may be defined as the range of the medical parameters of the patient that are not within the predefined normal range of the patient. Deviation range may be higher or lower than normal range, e.g., medical parameters that may be above and/or below the range that is defined as “normal”. For example, if a “normal” range of the pulse rate of the patient is defined as 50-150 beats per minute (BPM), and the sensed pulse is higher than 150 BPM or lower than 50 BPM, the pulse of the patient may be defined as being in the deviation range. When measurements are within the deviation range, the wearable device 105 may alert the patient and/or transmit the measurements to MC server 110.

The risk range may be defined as the range where certain parameter values indicate a condition that may be potentially dangerous to a particular patient or to a group of patients. In some embodiments, readings of parameter values in the risk range may automatically initiate an Emergency mode of operation. When measurements are within the risk range, the wearable device 105 may alert the patient and/or may transmit the measurements to MC server 110.

In accordance with some embodiments of the present invention, the staff of MC server 110 or the MC server 110 itself, may execute or perform software updates, which may include, for example, changes in diagnostic ranges, feature updates, changes in wearable device operation, or changes to other functions, for an individual patient's device 105 or for a group of devices 105. In some embodiments software updates may be remotely programmed into the patient's wearable device 105.

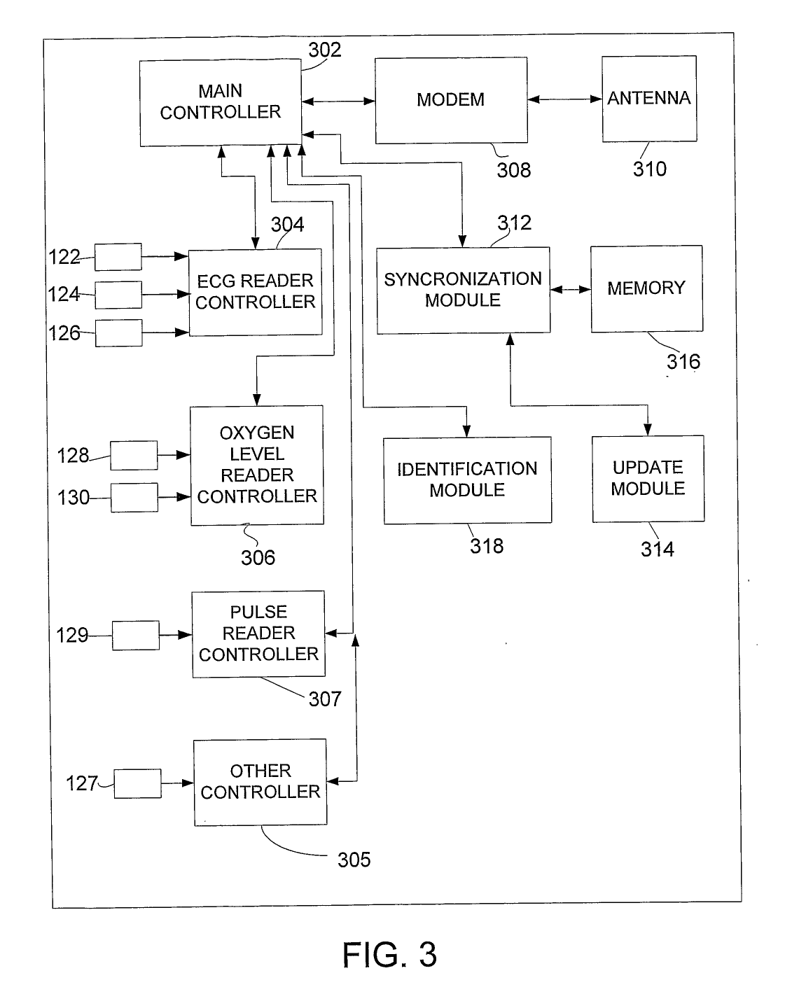

Reference is now made to FIG. 3, which is a schematic illustration of an internal layout of wearable device 105 in accordance with some exemplary embodiments of the present invention. Wearable device 105 may include, for example, a main controller 302 to control wearable device operation. Wearable device 105 may include an ECG reading controller 304 that may receive input from, for example, ECG electrodes 122, 124, and/or 126 (also shown in FIGS. 2A, 2B, and 2C, respectively), or from other sensors or combinations of sensors, and may generate output signals through main controller 302. Wearable device 105 may include a blood oxygen reading controller 306 that may receive input from, for example, SpO2 transceiver 128 and pulse transceiver 130, or from other sensors or combinations of sensors, and may generate output signals through main controller 302. Wearable device 105 may include a pulse reading controller 307 that may receive input from pulse sensor 129, or from other transceivers or sensors, or combinations of transceivers or sensors, and may generate suitable output signals through main controller 302. Wearable device 105 may include additional or alternative controllers 305, for example a blood pressure reading controller, blood sugar reading controller, temperature reading controller, Cardio Impedance (CI) reading controller, etc., that may receive input from one or more suitable sensor(s) 127, or from other transceivers or sensors, or combinations of transceivers or sensors, and may generate suitable output signals through main controller 302. Wearable device 105 may further include at least one modem 308, to transmit and receive data to and from MC server 110, for example using at least one antenna 310. Wearable device 105 may include one or more of a synchronization module 312, an update module 314, a memory module 316, and an identification module 318. Identification module 318, which may be used, for example, for MC to identify a particular device user, may include, for example, a Subscriber Identity Module (SIM) card and/or alternative identification means.

In some embodiments, main controller 302 may receive data from input components, for example, data received from functional buttons 112 and 114, emergency buttons 116 and 118, On/Off button 125, and/or from other components, such as service connector 138, charge connector 140, and battery 142. Main controller 302 may generate output that may be transferred to output components, for example, display area 134, modem 308, antenna 310, etc.

In some embodiments, ECG controller 304 may receive signals indicative of vital signs of the patient from ECG RA finger electrode 122, ECG LA wrist electrode 124, and/or ECG REF wrist electrode 126. ECG reading controller 304 may receive data, for example via main controller 302, from functionality buttons 112 and 114, emergency buttons 116 and 118, or other suitable sources. ECG reading controller 304 may transfer data, for example, via main controller 302, to output components, for example, to display area 134, to speaker 136, to modem 308, etc.

In some embodiments, Oxygen Level reading controller 306 may receive signals indicative of vital signs and/or other physiological parameters of the user from sensor 128 and/or 130. Oxygen Level reading controller 306 may also receive instruction data, for example via main controller 302, from functionality buttons 112 and 114, emergency buttons 116 and 118, or other suitable sources. Oxygen Level reading controller 306 may transfer data, for example via main controller 302, to output components, for example display area 134, speaker 136, communications modem 308 etc.

In some embodiments, Pulse reading controller 307 may receive signals indicative of vital signs and/or other physiological parameters of the user from sensor 129, or other suitable transceivers or sensors. Pulse reading controller 307 may receive data, for example via main controller 302, from functionality buttons 112 and 114, emergency buttons 116 and 118, or other suitable sources. Pulse Level controller 307 may transfer data, for example via main controller 302, to output components, for example display area 134, speaker 136, and communications modem 308 etc.

In some embodiments, wearable device 105 may include sensors and controllers to enable measurement and usage of blood pressure data, skin temperature data, body temperature data, respiration data, cardio impedance data, humidity level data and other suitable data. Respective controllers may receive signals indicative of vital signs and/or environmental parameters associated with the patient from respective sensors. Respective controllers may receive data, for example via main controller 302, from functionality buttons 112 and 114, emergency buttons 116 and 118, or other suitable sources. Respective controllers may transfer data, for example via main controller 302, to output components, for example display area 134, speaker 136, communications modem 308 etc.

In some embodiments, one or more of main controller 302, ECG reading controller 304, Oxygen level reading controller 306 and pulse reading controller 307, as well as other controllers 305, for example blood pressure reading controller, blood sugar reading controller, temperature reading controller etc. may be implemented in at least a single controller or in multiple separate controllers or combinations of controllers.

According to some embodiments of the present invention software updates, for example, including new values of the diagnostic ranges, may be stored temporarily in memory 316. Memory 316 may include, for example, one or more read-only memories (ROM), random access memories (RAM), electrically programmable read-only memories (EPROMs), electrically erasable and programmable read only memories (EEPROMs), FLASH memories, magnetic or optical cards, or any other types of media suitable for storing electronic instructions and values, or combinations thereof.

In accordance with some embodiments of the present invention, wearable device 105 may start using the software updates after their validity has been checked. In other embodiments wearable device 105 may start using the software updates after rebooting of device 105, after device 105 has been turned off and back on, or after a user's approval.

In accordance with some embodiments of the present invention, wearable device 105 may start the required parameter measurement after the validity of the request has been verified. In other embodiments wearable device 105 may take the required measurements immediately upon receipt of such request(s). In other embodiments wearable device 105 may take the required measurements after a pre-defined interval following receipt of such request(s).

After checking the validity of the software update, in accordance with some embodiments of the present invention, wearable device 105 may alert MC server 110 that an update either failed or succeeded. Such an alert may be sent by a SMS message, or using switched circuit communication, or by logging in to a computer associated with MC server 110 and uploading a message, for example, a warning or success message, and/or by initiating a data transfer, and/or by any other suitable means as are known in the art. Such a message or data transfer may be uploaded through the Internet, through Internet server 120, through the cellular network, or using any other suitable data communication mediums as are known in the art.

In accordance with some embodiments of the present invention, wearable device 105 may inform the patient through one or more output components, for example display area 134 and/or speaker 136, when the update is completed and/or whether the update was successful or not.

In some embodiments of the present invention, modem 308 may transfer and receive data from and to MC server 110, and/or from and to other devices, for example, via antenna 310. For example, modem 308 in association with antenna 310 may receive instructions sent from MC server 110 (e.g., through an SMS channel), or answer to voice calls received from MC server 110. Modem 308 may receive new software updates, for example including updated medical parameters, and updated diagnostic ranges, etc. Modem 308 may receive instruction data, for example, sensed measurements of vital signs, from main controller 302. Modem 308 may receive and transfer signals from and to microphone 132, identification module 318, and speaker 136. Modem 308 may be a wireless modem, or of another suitable technology enabling data transmission from or to wearable device 105.

In some embodiments of the present invention, data and signals transferred between the components and modules of wearable device 105 may be transferred in serial communication lines, I/O lines, and/or designated lines. For example, a V_{BAT} signal may activate an alert indicating that battery 142 is weak, and a V_{CHARGER} signal may activate an alert indicating that battery 142 is charged.

In some embodiments, synchronization module 312 may receive data from various components in wearable device 105, and may synchronize the data before transferring it to main controller 302. For example, synchronization module 312 may receive data from update module 314, memory unit 316 and/or identification module 318, and may determine, for example, which data is the most updated, and may initiate transfer of the most updated received data to main controller 302. In some embodiments, synchronization module 312 may be implemented as software and/or hardware components in the main controller 302. In some other embodiments of the present invention the update module 314 may be implemented as software and/or hardware components in the main controller 302. In some other embodiments of the current innovation the identification module 318 may be implemented as software and/or hardware components in the main controller 302. In some other embodiments of the present invention the memory 316 may be implemented as part of the main controller 302.

Wearable device 105, according to some embodiments of the present invention, may be remotely updated, for example, by updating the device software. Software updates may be initiated in accordance with previous measurements received from the wearable device within the MC server 110, and/or by the sole initiative of the MC. For example, wireless device 105 may include a plurality of sensors, one or more of which may be required for a particular patient and/or at a particular time. The remote reconfiguration or wireless update may, for example, enable a remote MC to determine which sensors are to be operated during selected periods, remotely configure the monitoring thresholds or ranges of selected sensors for an individual patient's parameters, customize one or more modes of operation for each patient, customize timing of parameter measurements, customize alert functions, determine of types of measurements to be monitored, customize diagnostic ranges, add new features or software improvements, delete features that are not relevant for a particular patient's condition monitoring, customize operational modes, correct software problems, and/or any other suitable modifications. Requests for measurements may include, for example, a request for new or updated pulse measurement, a new or updated ECG measurement, a new or updated blood oxygen saturation measurement, a command for a message to be displayed to the user, or other suitable requests for functions. Additionally or alternatively, the system may initiate requests for voice conversation with the user of device 105 or any other suitable requests.

The update may be initiated by the MC staff, for example, medical staff, information technology staff and/or technical/engineering staff, or a device at the MC server 110 may automatically update various parameters. The wearable device may have a bi-directional communication link with the MC server 110, which may be, for example, a clinic, hospital, remote center, medical professional, call center, or any other suitable provider of suitable medical services.

The remote reconfiguration may be performed, for example, by transmitting software updates, commands and/or instructions from MC server 10 to wireless device 105, using infrared line of sight, cellular, microwave, satellite, packet radio and/or spread spectrum technologies, or other suitable wireless data communication technologies. For example, a wireless data update of device 105 may be implemented using cellular network technology, for example, Global System for Mobile Communications (GSM), General Packet Radio Service (GPRS), Code Division Multiple Access (CDMA), Frequency Division Multiple Access (FDMA), Time Division Multiple Access (TDMA), Cellular Digital Packet Data (CDPD), Universal Mobile Telecommunication System (UMTS) or other suitable cellular communications methods. Additionally or alternatively, a remote update may be performed using short-range wireless communication protocols, for example, Wireless Fidelity (WiFi) and/or Bluetooth and/or ZigBee etc. Software updates may, for example, be packed into one or more update files that may be transmitted via SMS, FTP or other suitable protocols.

In accordance with some embodiments of the present invention, wearable device 105 may inform the user through one or more output components, for example display area 134 and/or speaker 136 etc., when an update is received and/or whether the implementation of the update was successful or not. For example, Modem 508 may receive data, for example sensed measurements of vital signs, from main controller 502. Modem 508 may receive and transfer signals from and/or to microphone 132, identification module 518, and speaker 136. Modem 508 may be a wireless modem, or another suitable technology for enabling data transmission from or to wearable device 105.

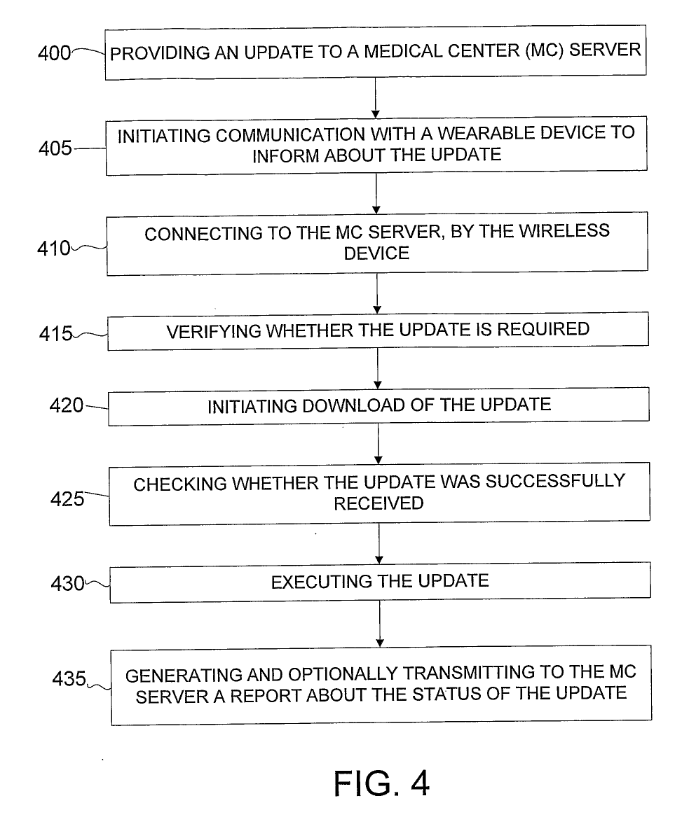

Reference is now made to FIG. 4, which schematically illustrates a method of remotely updating wearable device 105, in accordance with some embodiments of the present invention. At block 400, updates (e.g., software update, new commands, instructions, or new measurement thresholds etc.) may be provided by a medical team, a programming team and/or other technical team at MC server 110 to a computer associated with MC server 110. At block 405 MC server 110 may initiate communication with wearable device 105, for example, by sending a message to alert wearable device 105 or the patient wearing device 105 that an update is available and/or that an update may be necessary. In some embodiments this message may include, for example, the update's level of importance, preferred deadline or timing for downloading, etc. At block 410, wearable device 105 may connect to MC server 110 or to a suitable external device, such as a computer, through wire-based and/or wireless mediums. At block 415, MC may verify whether the particular update(s) are required for an individual patient. Such verification may be reached, for example, by the patient and/or MC staff, according to preconfigured criteria, after the patient has a medical examination, or from an analysis of monitored data transferred to MC server 110.

At block 420 wearable device 105 may communicate with MC server 110 to initiate downloading of the software update. The communication between wearable device 105 and MC server 110 may be done, for example, manually at the discretion of the patient, automatically (e.g., immediately, or after a period of time following receipt of the report about the available update, or after turning on the wearable device 105 etc. Wearable device 105 may communicate with Internet server 120, SMS transceiver 115 (both shown in FIG. 1), or other suitable data communication mediums to download the software update. At block 425, for example after the download of the diagnostic update is completed, wearable device 105 may check whether the software update was successfully received, for example, checksum tests, Cyclical Redundancy Checking (CRC), or any other software validation methods as are known in the art. Software verification may be used, for example, to verify the new ranges and/or to verify that the downloaded software is the correct software, that the software update has reached the correct destination, that the software update is the correct version, and/or that the software update has arrived completely and successfully.

At block 430, wearable device 105 may execute the software updates, for example, by updating new rules of operation, defining updated diagnostic ranges of the wearable device 105 etc. Software updates, for example, updateable parameter ranges, may be stored in memory 316 (shown in FIG. 3), such that when new parameter ranges are received, these new ranges may replace or modify the previously stored ranges in memory 316. The software updates may update and/or replace previous software version in memory 316. Executable files may be received, for example as attachments, and wearable device 105 may have suitable software for running received software updates. A message may be sent from device 105 to MC server 110 to inform MC server 110 that an update has been successfully implemented. At block 435, a report or message about the update may be generated by wearable device 105 and optionally transferred from wearable device 105 to MC server 110. A report or message may include, for example, a message or alert that a new update was received, executed and/or failed etc. The transfer of this report may be done, for example, by sending an SMS message from wearable device 105 to MC server 110, or by transferring data via a switched circuit communication link or cellular communication link, etc.

In accordance with some embodiments of the present invention, a SMS message sent from MC server 110 to wearable device 105 may include a software update, for example, including the values of new diagnostic ranges. In some embodiments the software update may include a reporting message. The updated software may be included in the reporting message, or in one or more separate SMS messages. Alternatively, when a communication session between MC server 110 and wearable device 105 is initiated, the staff of MC server 110 or an automatic procedure at MC server 110 itself may send the updated software in a single message or in multiple messages.

According to some embodiments of the present invention the updated software, for example, updated diagnostic values, may be encrypted by a computer associated with MC server 110, or in computer that is part of an automated portion of MC server 110, before transferring the updates to wearable device 105. Wearable device 105 may decrypt the updates and check their validity. Any method of encryption may be applied, for example, DES, 3DES, AES or other suitable methods.

In accordance with some embodiments of the present invention, MC server 110 may initiate an authentication process when wearable device 105 approaches or attempts to connect to a computer associated with MC server 110 to download a software update. For example, a Secure Sockets Layer (SSL) session or other suitable methods may be used to authenticate the data communication between MC server 110 and wearable device 105.

Some embodiments of the present invention are directed to an improved wearable device, a system, and a method for remotely initiating measurements, for example, initial and/or updated measurements of selected physiological and/or environmental parameters by the wearable device, as is described in detail below. The wearable device 105, according to some embodiments of the present invention, may be remotely requested to take measurements of physiological and/or environmental parameters, or to initiate other suitable functions. Such requests, which may be software updates, may be made in accordance with previous measurements received from the wearable device within the MC server 110. The request may be done by the MC staff, for example, medical staff, information technology staff and/or technical/engineering staff, or a device at the MC server 110 may automatically update various parameters. The wearable device may have a bi-directional communication link with the MC server 110, which may be, for example, a clinic, hospital, remote center, medical professional, call center, or any other suitable provider of suitable medical services. For example, wearable device may communicate with the MC server 110 using wireless data communication, for example cellular network technology, for example, Global System for Mobile Communications (GSM), General Packet Radio Service (GPRS), Code Division Multiple Access (CDMA), Frequency Division Multiple Access (FDMA), Time Division Multiple Access (TDMA), Cellular Digital Packet Data (CDPD), Universal Mobile Telecommunication System (UMTS) or other suitable cellular communications methods. Additionally or alternatively, a remote update may be performed using short-range wireless communication protocols, for example, Wireless Fidelity (WiFi) and/or Bluetooth, etc. Measurement requests may, for example, be packed into one or more update files that may be transmitted via SMS, FTP or other suitable protocols.

According to some embodiments of the present invention, the medical staff at MC 110 and/or an automatic procedure at MC 110 may request measurements, e.g., updated measurements and/or new measurements of physiological and/or environmental parameters (e.g., vital signs, body temperature), for example, in addition to or in place of the regular measurements performed according to the above-described modes of operation of the system. This may enable the system to initiate new or updated measurements at MC 110 by remotely controlling the operation of wearable device 105. Requests for measurements may include, for example, a request for new or updated pulse measurement, a new or updated ECG measurement, a new or updated blood oxygen saturation measurement, a command for a message to be displayed to the user, or other suitable requests for functions. Additionally or alternatively, the system may initiate requests for voice conversation with the user of device 105 or any other suitable requests. The measurement request(s) and/or other software updates may be transmitted directly to wearable device 105 using wire-based data communications and/or wireless data communications. In this way updated measurements and/or other actions may be requested from device 105, optionally remotely, by MC 110. For example, MC 110 may remotely initiate measurement of one or more parameters by wearable device 105, optionally for each individual user. For example, the medical or technical staff at MC 110 or a device at MC 110 may initiate diagnostic changes to help determine a user's status, for example, to enable remote testing of the user's physiological and/or environmental parameters. Desired new or updated ranges, commands, etc., may be determined for each user by the medical staff, and may be programmed remotely into the wearable device 105 using wireless data communications.

After checking the validity of the request for new and/or additional measurements, in accordance with some embodiments of the present invention, wearable device 105 may alert MC 110 that the request either failed or succeeded. Such an alert may be sent in an SMS message, by using switched circuit communication, by logging into a computer associated with MC 110 and uploading a message, for example a warning or success message, and/or initiating a data transfer etc. Such a message or data transfer may be uploaded using the Internet, via an Internet server 120, through a cellular network, and/or by using other suitable data communication mediums.

In accordance with some embodiments of the present invention, wearable device 105 may inform the user through one or more output components, for example display area 134 and/or speaker 136 etc., when a new and/or additional request for a measurement is received and/or whether the validity of the request was successful or not.

In some embodiments of the present invention, communications modem 508 may transfer and receive data from and to a MC 110, and/or from and to other devices, for example, via antenna 510. For example, modem 508 may receive instructions sent from MC 110 through the SMS channel, or answer to voice calls received from MC 110. Modem 508 may download new measurement requests, for example including updated measurement schedules, measurement types to be initiated, medical parameters, and updated diagnostic ranges, etc. Modem 508 may receive data, for example sensed measurements of vital signs, from main controller 502. Modem 508 may receive and transfer signals from and to microphone 132, identification module 518, and speaker 136. Modem 508 may be a wireless modem, or another suitable technology for enabling data transmission from or to wearable device 105.

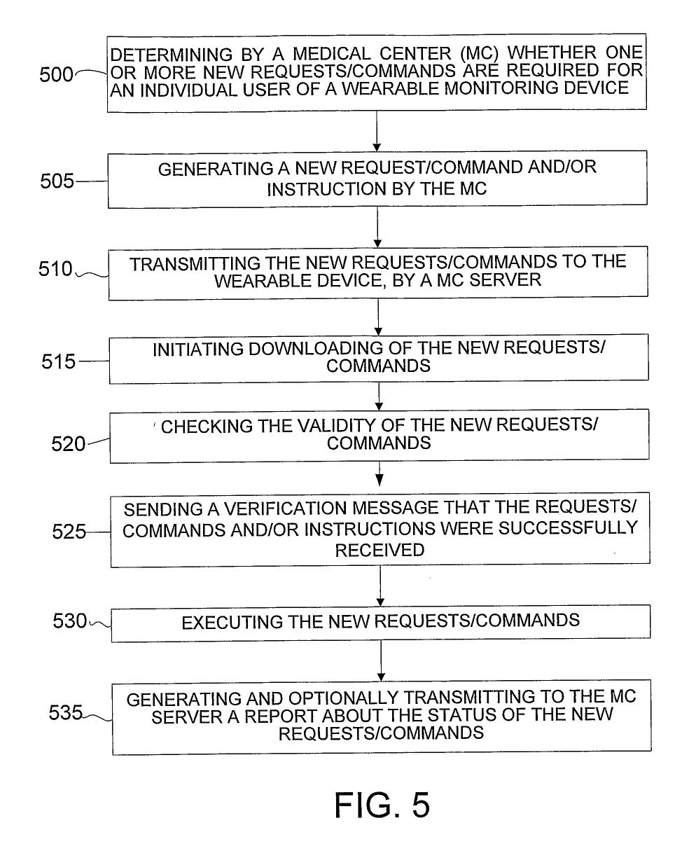

Reference is now made to FIG. 5, which schematically illustrates a method of remotely initiating updates (e.g., temporarily and/or permanent updates) to device 10 operations, for example, remotely initiating new requests/commands and/or new instructions in wearable device 105, in accordance with some embodiments of the present invention. At block 500, the MC may determine whether one or more requests or actions may be required for an individual user of wearable device 105. Such determination may be reached, for example, after the user has had a medical examination, or from an analysis of monitored occurrences that were transferred to MC server 110, etc. At block 505, after determining that, for example, one or more selected measurements are required, one or more requests, commands, instructions etc. relating to the required new and/or updated actions may be generated by MC server 110. A request may be embodied, for example, in a report or message. One such report or message may contain more than one request. Such a report or message may include, for example, an alert that a new request for measurement is ready, data about the new request (e.g., the request's level of importance), preferred deadline or timing for downloading, the content of the request (e.g., instructions, code, algorithms etc.) etc. The request, report and/or message generation may be implemented by the MC medical staff, or automatically by appropriate hardware and/or software at the MC, for example, based on trends of measurements from the last week, month, etc. At block 510 the report or message or the detailed new request/command and/or new instruction may be transferred to wearable device 105, for example, by sending an SMS message from MC server 110 to wearable device 105, by transferring data via a switched circuit communication link or cellular communication link etc. or by other suitable means.

At block 515, once wearable device 105 receives a message or other communication that a new action request, for example, a request to take additional or new measurements, is available, wearable device 105 may communicate with MC server 110 to initiate downloading of a new request/command and/or new instruction, which may, for example, include one or more measurement requests. The communication between wearable device 105 and MC server 110 may be done, for example, manually at the discretion of the user, or automatically (e.g., with a delay or without a delay) after receiving the report about the available request/command and/or instruction etc. Wearable device 105 may communicate with Internet server 120, SMS transceiver 115 (both shown in FIG. 1), or other suitable data communication mediums to download the update(s), if necessary.

At block 520, for example after the download of an update is completed, wearable device 105 may check the validity of the new request/command and/or new instruction by using, for example, checksum tests, Cyclical Redundancy Checking (CRC) etc. Software verification may, for example, be used to verify the validity of the new request/command and/or new instruction and/or to verify that the downloaded new request/command and/or new instruction has reached the correct destination and/or that it has arrived complete etc. At block 525, a message may be sent from device 105 to MC server 110 to inform MC server 110 that new requests/commands and/or new instructions have been successfully received.

At block 530, wearable device 105 may execute the new request(s)/command(s) and/or new instruction(s), thereby enabling implementation of the new instructions or requests by wearable device 105. For example, a request/command and/or instruction may include a command to initiate one or more measurements by the wearable device 105, select sensor activities etc. At block 535 a message may be generated and sent from device 105 to MC server 110 to inform MC server 110 that a new request/command and/or new instruction has been successfully executed (or failed).

In accordance with some embodiments of the present invention, an SMS message sent from MC server 110 to wearable device 105 may include one or more new and/or additional instructions, for example, requests for measurements. In some embodiment the request for additional measurement may include a reporting message. The request may be included in the reporting message, or in one or more separate SMS messages. Alternatively, when a communication session between MC server 110 and wearable device 105 is initiated, the staff of MC server 110 or MC system automatically, may send the request in a single or in multiple messages.

According to some embodiments of the present invention the request for new and/or updated measurements, for example, an urgent request for a new ECG measurement, may be encrypted by appropriate hardware and/or software on a computer associated with MC server 110, or elsewhere at MC server 110, before transferring the request to wearable device 105. Wearable device 105 may decrypt the request and check its validity. Device 105 may further encrypt the measurement(s) taken before sending the measurement(s) back to MC server 110. Any method of encryption may be applied, for example, DES, 3DES, AES or other suitable methods.

In accordance with some embodiments of the present invention MC server 110 may initiate an authentication process when wearable device 105 approaches or connects to a computer associated with MC server 110. For example, a Secure Sockets Layer (SSL) session or other suitable method may be used to authenticate the data communication between MC server 110 and wearable device 105.

According to some embodiments of the present invention, systems and methods are provided for enabling data privacy using authentication and/or encryption technologies when using mobile monitoring device 105. A mobile monitoring device 105, for example a wearable or portable medical device, may be required to follow the data privacy requirements and recommendations as described by, for example, the European Legislation (e.g.: Directive 95/46/EC and Directive 97/66/EC), USA legislation (HIPPA), and other common data privacy and security requirements.

Suitable data privacy and security requirements may include, for example, encryption of data, authentication of the user that may log into the system, using special protocols (known just to the system developers/users or any suitable commercial protocols or mechanisms) and any combination of the above.

In one example, all the data that is transferred between the mobile monitoring device 105 and a medical center or remote medical center (RMC) may be encrypted before transmission. The data may be encrypted using encryption key. The method of encryption (the encryption algorithm) may include one or more of: DES, triple DES, AES, RAS, or other suitable methods. The method may be publicized or non-publicized.

The data may be transmitted via a SMS mechanism. For example, GSM, GPRS, CDMA or any other cellular method may be used. After the transmission, the RMC may decrypt the data and continue its activities as needed based on this data and/or other tasks.

In a second example, as described above with regard to example 1, the transmission may be implemented by creating a call between the mobile monitoring device 105 and the RMC by the mobile monitoring device 105 (e.g., using switched circuit communications) over a cellular network.

In a third example, as described above with regard to example 1, the transmission may be implemented via a cellular network and the Internet network.

In a fourth example, as described above with regard to example 1, the transmission may be implemented using any other wireless communication technology.

In a fifth example, all the data that is transferred between the RMC and the mobile monitoring device 105 may be encrypted before transmission. The data may be encrypted using at least one encryption key. The method(s) of encryption (the encryption algorithm) may include one or more of: DES, triple DES, AES, RAS, or other suitable methods. The method may be publicized or non-publicized. The data may be being transmitted via a SMS mechanism, for example, using GSM, GPRS, CDMA or any other cellular method. After transmission of data the mobile monitoring device 105 may decrypt the data and continue its activities as needed based on this data and/or other requirements.

In a sixth example, as described above with regard to example 5, the transmission may be implemented by creating a call between the RMC and the mobile monitoring device 105 by the RMC (e.g., using switched circuit communications) over the cellular network.

In a seventh example, as described above with regard to example 5, the transmission may be implemented via the cellular network and the Internet network.

In an eighth example, as described above with regard to example 5, the transmission may be implemented in any other wireless communication method.

In a ninth example, any combination of examples 1, 2, 3 or 4 with examples 5, 6, 7 or 8 may be implemented.

According to some embodiments of the present invention, system 100 may enable authentication of transmitted data. In one example, when the mobile monitoring device 105 approaches the RMC to create a call between the mobile monitoring device 105 and the RMC by the mobile monitoring device 105 (e.g., using switched circuit communications) over the cellular network (e.g., GSM, GPRS, CDMA or any other cellular method) an authentication requirement may be applied to prevent un-authorized device to log to the RMC. The authentication may be implemented using, for example, a public key. The authentication process may use SSL techniques or any other publicized or non-publicized techniques.

In a second example, as described above with regard to example 1, the transmission may be implemented via the cellular network and/or the Internet network.

In a third example, as described above with regard to example 1, the transmission may be implemented using any other wireless communication method.

In a fourth example, examples 1, 2 or 3 may be implemented, but the key used for the authentication may be a private key.

According to some embodiments of the present invention, system 100 may enable the hiding of user identification. In one example, in order to prevent the correlation of the user with the personalized medical (or any other information) exchanged between the mobile monitoring device 105 and the RMC (in either of the directions, or in both directions), both the mobile monitoring device 105 and the RMC may not transmit any form of public identification of the mobile monitoring device 105 user (e.g., ID number, social security number etc.). The substitute may be, for example, a special ID known just to the mobile monitoring device 105 and RMC.

In a second example, user identification may be hidden, as described in example one, however the special ID may be changed at pre-defined or random periods of time. This ID data may be exchanged, for example, using or without using encryption and/or authentication.

According to some embodiments of the present invention, system 100 may be operated using special protocols. In one example, all the data (or part of the data) that is being transmitted between the mobile monitoring device 105 and the RMC (in either direction, or both directions) may use predefined protocols known just to the mobile monitoring device 105 and the RMC to exchange data.

In a second example, all the data (or part of the data) that is being transmitted may use predefined codes for the messages (e.g., the transmission may include only a code for the message and not the message content).

In a third example, the data may be transmitted as described in example 2, but the special ID may be changed at pre-defined or random periods of time. The exchange of ID data may or may not use encryption and/or authentication.

In addition, any combination of the above-described techniques may be implemented.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

What is claimed is:

1. A system for monitoring parameters, comprising a medical center server enabled to remotely reconfigure the functioning of a wireless mobile monitoring device, said device including an array of sensors.

2. The system of claim 1, wherein the functioning of said array of sensors may be remotely customized by said server.

3. The system of claim 1, wherein said array of sensors includes at least one physiological sensor.

4. The system of claim 1, wherein said array of sensors includes a plurality of physiological sensors.

5. The system of claim 1, wherein said array of sensors includes a plurality of environmental sensors.

6. The system of claim 1, wherein said array of sensors includes at least one physiological sensor and at least one environmental sensor.

7. The system of claim 1, wherein said monitoring device is to perform one or more functions selected from the group consisting of measuring parameters, transmitting parameter data, processing parameter data, analyzing parameter data, initiating device actions, updating parameter settings, providing warnings, and providing instructions.

8. The system of claim 1, wherein said monitoring device is to function in one or more of keeper mode, extended mode, and emergency mode.

9. The system of claim 1, wherein said monitoring device is to measure one or more selected parameters continuously and/or intermittently.

10. The system of claim 1, wherein said monitoring device is to automatically send a warning message to said medical center server if parameters measured exceed a selected threshold.

11. The system of claim 1, wherein said remote configuration of said mobile monitoring device includes remotely implementing a customized software update.

12. The system of claim 1, wherein communications between said monitoring device and said medical center are encrypted.

13. The system of claim 1, wherein communications between said monitoring device and said medical center are authenticated.

14. The system of claim 1, wherein communications between said monitoring device and said medical center are encrypted and authenticated.

15. The system of claim 1, comprising a synchronization module.

16. The system of claim 1, comprising an update module.

17. The system of claim 17, comprising a data encryption module and a data authentication module.

18. A device for monitoring of parameters, the device comprising:
an array of sensors, each sensor having a sensor controller; and
a main controller to enable reconfiguration of said sensor controllers by commands received from a remote server.

19. The device of claim 18, wherein said array of sensors includes at least one sensor to measure physiological parameters and at least one sensor to measure environmental parameters.

20. The device of claim 18, wherein said array of sensors includes one or more sensors selected from the group consisting of an ECG sensor, Oxygen level sensor, pulse sensor, blood pressure sensor, SpA₂ sensor, glucose level sensor, sweat sensor, skin temperature sensor, pH level sensor, external temperature sensor, air humidity level sensor, and pollution level sensor.

21. The device of claim 18, comprising a synchronization module.

22. The device of claim 18, comprising an update module.

23. The device of claim 18, comprising a data encryption module.

24. The device of claim 18, comprising a data authentication module.

25. The device of claim 18, comprising a data encryption module and a data authentication module.

26. A method for remotely reconfiguring a monitoring device, the method comprising:
transmitting commands to a wireless monitoring device, from a medical center server, to remotely reconfigure settings of said device; and
reconfiguring settings of said device, by a main controller in said wireless monitoring device.

27. The method of claim 26, comprising checking the validity of said commands.

28. The method of claim 26, wherein said wireless monitoring device includes an array of sensors, said sensors enabled to be individually reconfigured by said medical center server.

29. The method of claim 26, wherein said array of sensors includes one or more sensors selected from the group consisting of an ECG sensor, Oxygen level sensor, pulse sensor, blood pressure sensor, SpA₂ sensor, glucose level sensor, sweat sensor, skin temperature sensor, pH level sensor, external temperature sensor, air humidity level sensor, and pollution level sensor.

30. The method of claim 26, comprising remotely initiating one or more actions in said wireless monitoring device, by said medical center server.

31. The method of claim 26, wherein said remote configuration includes implementing customized software updates.

32. The method of claim 26, comprising remotely updating client software in said wireless monitoring device, by said medical center server.

33. The method of claim 26, comprising encrypting data communicated between said wireless monitoring device and said medical center server.

34. The method of claim 26 comprising authenticating data communicated between said wireless monitoring device and said medical center server.

35. The method of claim 26, comprising authenticating data and encrypting data communicated between said wireless monitoring device and said medical center server.
